(19) United States
(12) Patent Application Publication
(10) Pub. No.: US 2024/0123095 A1
(43) Pub. Date: Apr. 18, 2024

(54) CROSSLINKED RADIOPAQUE NETWORKS FOR MEDICAL APPLICATIONS

(71) Applicant: Boston Scientific Scimed, Inc., Maple Grove, MN (US)

(72) Inventors: Yen-Hao Hsu, Shrewsbury, MA (US); Joseph Thomas Delaney, JR., Minneapolis, MN (US)

(73) Assignee: Boston Scientific Scimed, Inc., Maple Grove, MN (US)

(21) Appl. No.: 18/469,762

(22) Filed: Sep. 19, 2023

Related U.S. Application Data
(60) Provisional application No. 63/410,154, filed on Sep. 26, 2022.

(51) Int. Cl. A61K 51/04; C07D 491/18

(52) U.S. Cl. CPC A61K 51/0446; C07D 491/18

(57) ABSTRACT

In some aspects, the present disclosure pertains to a system for forming a radiopaque product that comprises an iodinated compound comprising one or more diene containing moieties and a multi-arm polymer comprising a plurality of dienophile containing moieties, wherein the diene containing moieties of the iodinated compound couple with the dienophile containing moieties of the multi-arm polymer through a Diels-Alder reaction. Other aspects of the disclosure pertain to crosslinked networks that are formed by combining such an iodinated compound with such a multi-arm polymer, whereupon the diene containing moieties couple with the dienophile containing moieties through a Diels-Alder reaction. Still other aspects of the disclosure pertain to methods of treatment comprising administering to a subject a mixture that comprises such an iodinated compound and such a multi-arm polymer, whereupon the diene containing moieties couple with the dienophile containing moieties by undergoing a Diels-Alder reaction after administration.

### CROSS-REFERENCE TO RELATED APPLICATIONS

This application claims the benefit of U.S. Provisional Patent Application Ser. No. 63/410,154 filed on Sep. 26, 2022, the disclosure of which is incorporated herein by reference.

### FIELD

The present disclosure relates to iodinated compounds, to crosslinked radiopaque networks formed from iodinated compounds, and to methods of making and using iodinated compounds and networks, among other aspects. The iodinated compounds and networks of the present disclosure are useful, for example, in forming hydrogels for various medical applications.

### BACKGROUND

In vivo crosslinked hydrogels based on star-poly(ethylene glycol) (star-PEG) polymers functionalized with reactive ester end groups which are reacted with lysine trimer (Lys-Lys-Lys) as a crosslinker to rapidly form crosslinked hydrogels, such as SpaceOAR®, have become clinically significant materials as adjuvants in radiotherapies. See “Augmenix Announces Positive Three-year SpaceOAR Clinical Trial Results,” Imaging Technology News, Oct. 27, 2016.

Hydrogels in which some of the star-PEG branches have been functionalized, with 2,3,5-triiiodobenzamide (TIB) groups replacing part of ester end groups, such as SpaceOAR Vue®, have also been developed, which provide enhanced radiocontrast. See “Augmenix Receives FDA Clearance to Market its TraceIT® Tissue Marker,” BusinessWire Jan. 28, 2013. TraceIT® hydrogel remains stable and visible in tissue for three months, long enough for radiotherapy, after which it is absorbed and cleared from the body. Id.

While the above approach is effectual, using the arms of the star polymer to functionalize the hydrogel with iodine means that there are fewer arms available to crosslink. This can be overcome by adding more polymer, but the loading of solids increases, which can adversely impact viscosity. Reducing the molecular weight can cut down on the loading of solids, but this also can result in a lower melting point, and problems with processability. An additional effect of the reduced crosslink density per star polymer is that the resulting gel has a slower cure rate, which means the gel is liquid and mobile in vivo for longer time periods, opening up opportunities for unintended side-reactions and material displacement. Moreover, TIB is sparingly water soluble, meaning that there is an upper limit to how much iodine can be added before the solubility of the gel becomes impacted and it becomes difficult to form a smooth and consistent hydrogel. In the event that the concentration of TIB groups becomes so high that the star-PEG precipitates out of solution, the TIB groups can physically crosslink the system before it even reacts, requiring greater force to dispense. In addition, star PEG labeled with 2,3,5-triiiodobenzamide end groups often show discoloration from thermal degradation. While this doesn't impact their functionality, this is a cosmetic defect that is preferably avoided.

Furthermore, trilysine is utilized as a crosslinker to form a crosslinked hydrogel with TIB-functionalized star PEG. Unfortunately, amine-based biofluid is ubiquitous in vivo and acts as a natural source of crosslinker that competes with the trilysine, resulting in off-target crosslinking with less density in some cases. Finally, a buffer solution is added to the hydrogel precursor to maintain the pH value and avoid uncontrolled crosslinking conditions before injection, which increases the overall complexity of troubleshooting, manufacturing, and quality control.

For these and other reasons, an alternative strategy for an iodine-labelled crosslinked hydrogel with high reactive selectivity without any buffer solution is extremely desirable.

### SUMMARY

The present disclosure provides an alternative approach to that described above based on the Diels-Alder reaction.

In some aspects, the present disclosure pertains to a system for forming a radiopaque product that comprises (a) an iodinated compound comprising one or more diene containing moieties, more typically two or more diene moieties, and (b) a multi-arm polymer comprising a plurality of dienophile containing moieties, wherein the diene containing moieties of the iodinated compound couple with the dienophile containing moieties of the multi-arm polymer by undergoing a Diels-Alder reaction.

In some embodiments, the diene containing moieties are furan containing moieties and the dienophile containing moieties are maleimide containing moieties.

In some embodiments, which can be used in conjunction with any of the above aspects and embodiments, the iodinated compound comprises a core, one or more furan containing moieties attached to the core, and one or more iodine containing moieties attached to the core. In some of these embodiments, (a) the one or more furan containing moieties may be attached to the core though an ester group and the one or more iodine containing moieties may be attached to the core through an amide group and/or (b) the core may be a residue of a hydroxy acid compound, with the one or more furan containing moieties corresponding to residues of a carboxylic-acid-substituted furan containing compound and the one or more iodine containing moieties corresponding to residues of an amino-substituted iodinated compound and/or (c) the one or more iodine containing moieties may comprise an aromatic structure that is substituted with one or more iodine groups and, optionally, one or more hydrophilic functional groups, which may be, for example, selected from hydroxyl groups and C₁-C₄-hydroxyalkyl groups.

In some embodiments, which can be used in conjunction with any of the above aspects and embodiments, the multi-arm polymer comprises a plurality of hydrophilic polymer arms. In certain embodiments, the hydrophilic polymer arms may be formed from one or more hydrophilic monomers selected from ethylene oxide, N-vinyl pyrrolidone, oxazolines, hydroxyethyl acrylate, hydroxyethyl methacrylate, PEG methyl ether acrylate or PEG methyl ether methacrylate, or N-isopropylacrylamide. In certain embodiments, two or more hydrophilic polymer arms of the plurality of hydrophilic polymer arms may each comprise one or more dienophile end groups, in which case the dienophile end groups may be linked to the two or more hydrophilic polymer arms by a hydrolysable ester group.

In some embodiments, which can be used in conjunction with any of the above aspects and embodiments, the system comprises a first composition that comprises the iodinated compound comprising the one or more diene containing moieties in a first container and a second composition that comprises the multi-arm polymer comprising the plurality of dienophile containing moieties in a second container. For example, the first and second containers may be independently selected from vials and syringe barrels, among other contains. In certain embodiments, the system may further comprise a delivery device.

Other aspects of the present disclosure pertain to crosslinked networks formed by combining an iodinated compound comprising one or more diene containing moieties in accordance with any of the above aspects and embodiments with a multi-arm polymer comprising a plurality of dienophile containing moieties in accordance with any of the above aspects and embodiments, wherein the diene containing moieties of the iodinated compound couple with the dienophile containing moieties of the multi-arm polymer by undergoing a Diels-Alder reaction.

In some embodiments, the crosslinked network is a hydrogel.

In some embodiments, which can be used in conjunction with any of the above aspects and embodiments, the crosslinked network has a radiopacity that is greater than 250 Hounsfield units.

Still other aspects of the present disclosure pertain to methods of treatment comprising administering to a subject a mixture that comprises an iodinated compound comprising the one or more diene containing moieties in accordance with any of the above aspects and embodiments and a multi-arm polymer comprising the plurality of dienophile containing moieties in accordance with any of the above aspects and embodiments, such that the diene containing moieties of the iodinated compound couple with the dienophile containing moieties of the multi-arm polymer by undergoing a Diels-Alder reaction (and thus forming a Diels-Alder adduct) after administration.

Potential benefits associated with the present disclosure include one or more of the following: radiocontrast is maintained, highly selective crosslinking may be achieved thereby minimizing off-target crosslinking, buffer solutions may be avoided, the melting point of the solid components of the hydrogel can be maintained above 40° C. improving storage and handling, homogeneity of the final hydrogel may be improved, in vivo persistence may be obtained, and cure kinetics may be maintained.

The above and other aspects, embodiments, features and benefits of the present disclosure will be readily apparent from the following detailed description.

### BRIEF DESCRIPTION OF THE DRAWINGS

FIG. 1A schematically illustrates a method of adding furan containing moieties to a hydroxy acid, in accordance with an embodiment of the present disclosure.

FIG. 1B schematically illustrates a method of wherein an iodinated amino compound is coupled to the product of FIG. 1A, in accordance with an embodiment of the present disclosure.

FIG. 2A schematically illustrates a method of adding furan containing moieties to a hydroxy acid, in accordance with another embodiment of the present disclosure.

FIG. 2B schematically illustrates a method of wherein an iodinated amino compound is coupled to the product of FIG. 2A, in accordance with an embodiment of the present disclosure.

FIG. 3A schematically illustrates a method of adding a furan containing moiety to a hydroxy acid, in accordance with another embodiment of the present disclosure.

FIG. 3B schematically illustrates a method of wherein an iodinated amino compound is coupled to the product of FIG. 3A, in accordance with an embodiment of the present disclosure.

FIG. 4 schematically illustrates a method of adding maleimide containing moieties to a multi-arm polymer that comprises a core region and a plurality of polyethylene oxide (PEO) arms, in accordance with an embodiment of the present disclosure.

FIG. 5 schematically illustrates a method whereby the maleimide containing multi-arm polymer product of FIG. 4 is crosslinked with the furan containing iodinated product of FIG. 1B, according to an aspect of the present disclosure.

FIG. 6 schematically illustrates a method whereby the maleimide containing multi-arm polymer product of FIG. 4 is crosslinked with the furan containing iodinated product of FIG. 2B, according to an aspect of the present disclosure.

FIG. 7 schematically illustrates a method whereby the maleimide containing multi-arm polymer product of FIG. 4 is reacted with the furan containing iodinated product of FIG. 3B, according to an aspect of the present disclosure.

### DETAILED DESCRIPTION

In some aspects, the present disclosure pertains to radiopaque products that comprise a Diels-Alder reaction product of an iodinated compound comprising one or more diene containing moieties and a multi-arm polymer that comprises a plurality of dienophile containing moieties.

In some embodiments, the diene containing moieties are furan containing moieties. In some embodiments, the dienophile containing moieties are maleimide containing moieties.

In some embodiments, the iodinated compound comprises two or more diene moieties, in which case the reaction product may be a crosslinked reaction product. In some cases, the crosslinked reaction product is a hydrogel.

Although particular examples of iodinated compounds comprising one or more diene containing moieties and particular examples of multi-arm polymers that comprise a plurality of dienophile containing moieties are described herein, it will be appreciated that iodinated compounds comprising one or more dienophile containing moieties and multi-arm polymers that comprise a plurality of diene containing moieties may also be formed using suitable methods.

In various aspects, the present disclosure provides methods of forming iodinated compounds that comprise one or more diene containing moieties and one or more iodine containing moieties are provided. In some embodiments, iodinated compounds comprising one or more diene containing moieties can be formed based on hydroxy acid compounds that comprise one or more hydroxyl groups and one or more carboxylic acid groups. As will be seen from the discussion to follow, the hydroxyl group sites can be used to add furan containing moieties whereas the carboxylic acid group sites can be used to add iodine containing moieties. In order to act as a crosslinking agent, the iodinated compounds preferably comprise a plurality of diene containing moieties. Such multifunctional compounds can be formed from hydroxy acid compounds that comprise a plurality hydroxyl groups and one or more carboxylic acid groups.

Numerous hydroxy acid compounds that comprise one or more hydroxyl groups and one or more carboxylic acid groups can be used in conjunction with the present disclosure. A few examples of such compounds include 2,3-dihydroxy-butanedioic acid,

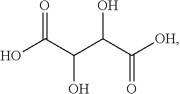

2,3,4-trihydroxy-pentanedioic acid,

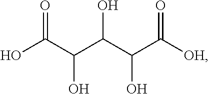 [SMILES: O=C(O)C(O)C(O)C(O)C(=O)O]

2,3,4,5-tetra hydroxyhexanedioic acid,

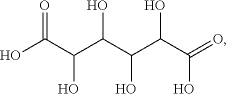 [SMILES: O=C(O)C(O)C(O)C(O)C(O)C(=O)O]

6-hydroxy-5,5-bis(hydroxymethyl)hexanoic acid,

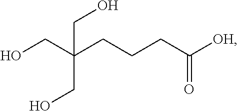 [SMILES: O=C(O)CCCC(CO)(CO)CO]

citric acid,

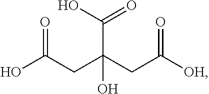 [SMILES: O=C(O)CC(O)(CC(=O)O)C(=O)O]

2,3-dihydroxypropanoic acid,

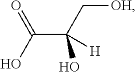

2,3,4-trihydroxybutanoic acid,

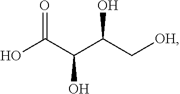

and 4-hydroxy-2,3-bis(hydroxymethyl)butanoic acid,

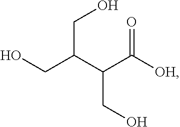 [SMILES: O=C(O)C(CO)C(CO)CO]

among many others. In some embodiments, hydroxy groups can be formed from polyacids by a suitable reduction reaction. For example, 2-hydroxy-2-hydroxyethyl-butanedioic acid, a dicarboxylic acid diol compound, can be formed by reduction of citric acid in the presence of lithium aluminum hydride (LiAlH₄):

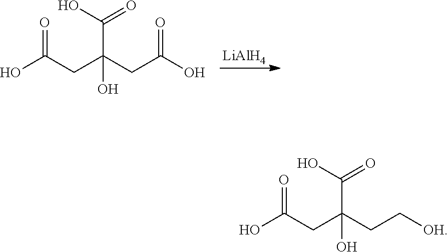

In a first step, an ester coupling reaction is performed between hydroxyl group(s) of a hydroxy acid compound that comprises one or more hydroxyl groups and one or more carboxylic acid groups (e.g., selected from one of those described above, among others) and a carboxylic acid group of a carboxylic-acid-substituted furan containing compound, for example, furan containing compound substituted with a C₁-C₆-carboxylic acid moiety, such as 3-(4-ethylfuran-2-yl) propanoic acid, 3-(furan-2-yl)propanoic acid, 2-(furan-2-yl)acetic acid, 4-(furan-2-yl)butanoic acid, 4-(4-ethylfuran-2-yl)butanoic acid, or 2-(4-ethylfuran-2-yl)acetic acid, among others.

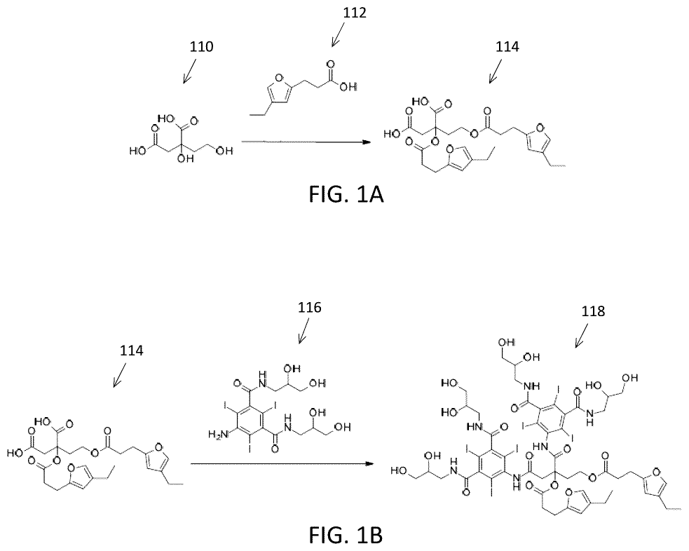

For instance, as shown in FIG. 1A, an ester coupling reaction may be performed between a carboxylic acid group of 3-(4-ethylfuran-2-yl) propanoic acid 112,

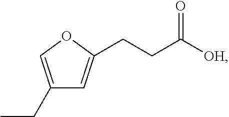

and hydroxyl groups of the dicarboxylic acid diol compound, 2-hydroxy-2-hydroxyethyl-butanedioic acid 110,

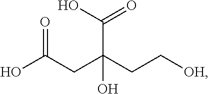 [SMILES: O=C(O)CC(O)(CCO)C(=O)O]

to form a multifunctional furan compound, specifically a difuran-functionalized diacid 114 having two ester bonds, for example, using a suitable coupling reagent such as dicyclohexylcarbodiimide (DCC), diisopropylcarbodiimide (DIC).

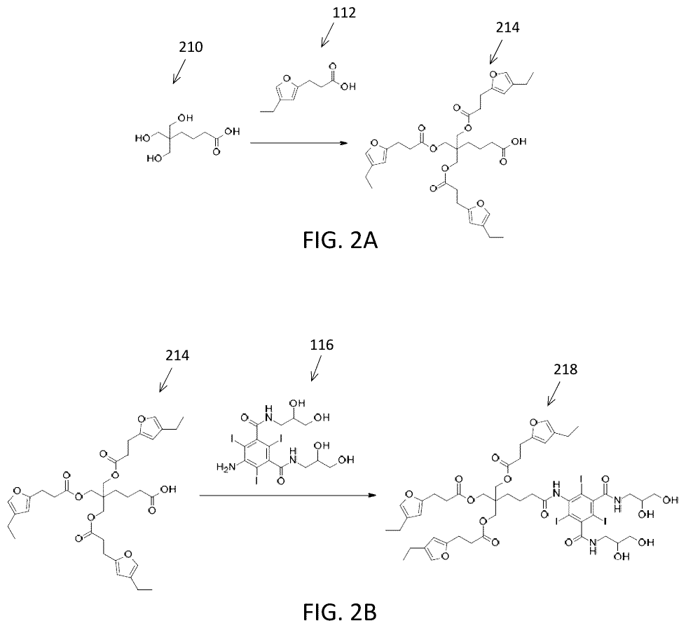

As another example, as shown in FIG. 2A, an ester coupling reaction may be performed between a carboxylic acid group of 3-(4-ethylfuran-2-yl) propanoic acid 112,

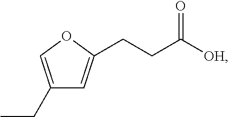

and hydroxyl groups of 6-hydroxy-5,5-bis(hydroxymethyl)hexanoic acid 210,

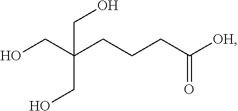 [SMILES: O=C(O)CCCC(CO)(CO)CO]

to form a multifunctional furan compound 214 having three ester bonds, for example, using a suitable coupling reagent.

In a second step, carboxylic acid groups in the multifunctional furan compound product of the first step are reacted with an amino group of an amino-substituted iodinated compound in an amide coupling reaction (e.g., using a suitable coupling reagent such as a carbodiimide coupling reagent).

For instance, the amino-substituted iodinated compound may be an amino-substituted iodinated aromatic compound comprising a monocyclic or multicyclic aromatic structure that is substituted with one or more iodine groups and an amino group. In some of these embodiments, the monocyclic or multicyclic aromatic structure may be further substituted with one or a plurality of hydrophilic functional groups. Such hydrophilic functional groups may be selected, for example, from hydroxyl groups, and hydroxyalkyl groups such as C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), among others. The hydroxyalkyl groups may be linked to the monocyclic or multicyclic aromatic structures directly or through any suitable linking moiety, which may be selected, for example, from amide groups, amine groups, ether groups, ester groups, or carbonate groups, among others.

For instance, in some embodiments, the amino-substituted iodinated compound may be a 5-amino-1,3-substituted-2,4,6-triiodobenzene compound, wherein a substituent at each of the 1- and 3-positions comprises a hydrophilic functional group, for example, a hydroxyalkyl group, which may be selected from those described above and which may be linked to the benzene structure directly or through any suitable linking moiety. In a particular example, the iodinated amino compound may comprise a 5-amino-1,3-hydroxyalkyl-substituted-2,4,6-triiodo-1,3-benzenedicarboxamide compound, for instance, a 5-amino-N,N′-bis(hydroxyalkyl)-2,4,6-triiodo-1,3-benzenedicarboxamide compound, such as 5-amino-N,N′-bis(2,3-dihydroxypropyl)-2,4,6-triiodo-1,3-benzenedicarboxamide, also known as 5-amino-N,N′-bis(2,3-dihydroxypropyl)-2,4,6-triiodoisophthalamide, in which the 5-amino group is used to form an amide linkage with the furan product of the first step.

In a particular embodiment shown in FIG. 1B, the carboxylic acid groups of the multifunctional furan product 114 of FIG. 1A are reacted with the amino group of 5-amino-N,N′-bis(2,3-dihydroxypropyl)-2,4,6-triiodoisophthalamide 116 (CAS #76801-93-9) in an amide coupling reaction in the presence of a carbodiimide coupling reagent, for example, 1-ethyl-3-(3-dimethylaminopropyl)carbodiimide hydrochloride (EDCHCI). The resulting iodinated multifunctional furan compound 118 contains six iodine groups and two furan groups and is suitable as an iodinated crosslinking agent for another molecule that comprises dienophile containing moieties (e.g., maleimide containing moieties) as described below.

In another particular embodiment shown in FIG. 2B, the carboxylic acid groups of the multifunctional furan product 214 of FIG. 2A are reacted with the amino group of 5-amino-N,N′-bis(2,3-dihydroxypropyl)-2,4,6-triiodoisophthalamide 116 in an amide coupling reaction in the presence of a carbodiimide coupling reagent, for example, 1-ethyl-3-(3-dimethylaminopropyl)carbodiimide hydrochloride (EDCHCI). The resulting iodinated multifunctional furan compound 218 contains three iodine groups and three furan groups and is suitable as a crosslinking agent for another molecule that comprises dienophile containing moieties (e.g., maleimide containing moieties) as described below.

Although not suitable for use as crosslinking agents for another molecule that comprises dienophile containing moieties, in further embodiments, iodinated compounds comprising a single diene containing moiety may be formed from hydroxy acid compounds that comprise a single hydroxyl group and one or more carboxylic acid groups. Numerous hydroxy acid compounds that comprise a single hydroxyl group and one or more carboxylic acid groups are available and include citric acid,

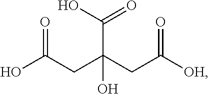 [SMILES: O=C(O)CC(O)(CC(=O)O)C(=O)O]

among many others.

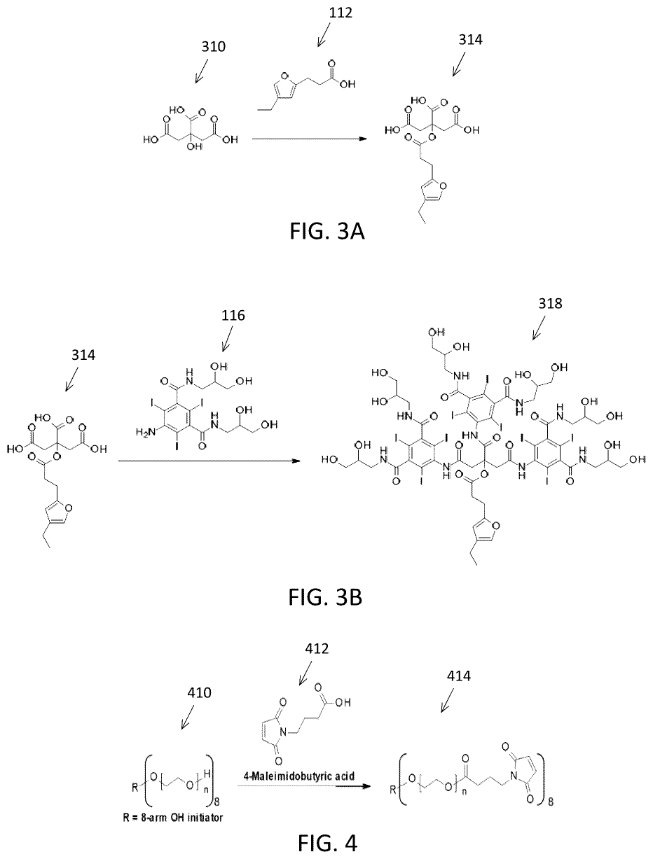

In a first step, an ester coupling reaction is performed between a carboxylic acid group of carboxylic-acid-substituted furan, such as one of those described above, among others, and the hydroxyl group of the hydroxy compound. For example, as shown in FIG. 3A, an ester coupling reaction may be performed between a carboxylic acid group of 3-(4-ethylfuran-2-yl) propanoic acid 112 and the hydroxyl group of citric acid 310 to form a monofunctional furan compound 314 having one ester bond, using a suitable coupling reagent such as dicyclohexylcarbodiimide (DCC), diisopropylcarbodiimide (DIC).

In a second step, the carboxylic acid group(s) in the monofunctional furan product of the first step are reacted with the amino group of an amino-substituted polyiodinated aromatic compound, for example, as described above, among others, in an amide coupling reaction (e.g., via a suitable coupling reagent). For instance, as shown in FIG. 3B, the three carboxylic acid groups of the monofunctional furan product 314 of FIG. 3A are reacted with the amino group of 5-amino-N,N′-bis(2,3-dihydroxypropyl)-2,4,6-triiodoisophthalamide 116 in an amide coupling reaction in the presence of a carbodiimide coupling reagent, for example, EDCHCI. The resulting iodinated monofunctional furan compound 318 contains nine iodine groups and one furan group and is useful, for example, as a modifier compound for introducing radiopacity to another molecule that comprises dienophile containing moieties (e.g., maleimide containing moieties) as described below.

Multi-arm polymers that comprise a plurality of dienophile containing moieties include those that comprise a plurality of polymer arms (e.g., having two, three, four, five, six, seven, eight, nine, ten, eleven, twelve or more arms), wherein two or more polymer arms of the multi-arm polymers each comprise one or more dienophile end groups. Examples of dienophile groups include of dienophile containing moieties that comprise maleimide groups,

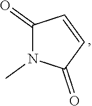

which will now be described.

In some embodiments, compositions containing the multi-arm polymers may be provided in which a percentage of the polymer arms comprising one or more dienophile containing moieties may correspond to between 50% and 100% of the total number of polymer arms in the composition (e.g., ranging anywhere from 50% to 70% to 80% to 90% to 95% to 99% to 100% of the total number of polymer arms). Typical average molecular weights for the multi-arm polymers for use herein range from 15 to 20 kDa, among other values. In various embodiments, the multi-arm polymers for use herein have a melting point of 40° C. or greater, preferably 45° C. or greater.

In various embodiments, the polymer arms are hydrophilic polymer arms. Such hydrophilic polymer arms may be composed of any of a variety of synthetic, natural, or hybrid synthetic-natural polymers including, for example, poly(alkylene oxides) such as poly(ethylene oxide) (PEO) (also referred to as polyethylene glycol or PEG), poly(propylene oxide) or poly(ethylene oxide-co-propylene oxide), poly(N-vinyl pyrrolidone), polyoxazolines including poly(2-alkyl-2-oxazolines) such as poly(2-methyl-2-oxazoline), poly(2-ethyl-2-oxazoline) and poly(2-propyl-2-oxazoline), poly(vinyl alcohol), poly(allyl alcohol), poly(hydroxyethyl acrylate), poly(hydroxyethyl methacrylate), poly(N-isopropylacrylamide) (PNIPAAM), polysaccharides, and combinations thereof.

In some embodiments, the polymer arms extend from a core region. In certain of these embodiments, the core region comprises a residue of a polyol that is used to form the polymer arms. Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, adonitol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

In certain beneficial embodiments, the core region comprises a residue of a polyol that contains two, three, four, five, six, seven, eight, nine, ten or more hydroxyl groups. In certain beneficial embodiments, the core region comprises a residue of a polyol that is an oligomer of a sugar alcohol such as glycerol, mannitol, sorbitol, inositol, xylitol, or erythritol, among others.

In certain embodiments, the dienophile containing moieties are linked to the polymer arms via hydrolysable ester groups.

Multi-arm polymers having arms that comprise one or more dienophile containing moieties can be formed, for example, from multi-arm polymers having polymer arms that comprise one or more hydroxyl end groups. For instance, an ester coupling reaction may be performed between a carboxylic acid group of a carboxylic-acid-substituted dienophile containing compound and hydroxyl groups of a multi-arm polymer having polymer arms that comprise one or more hydroxyl end groups, for example, using a suitable coupling reagent such as carbodiimide coupling reagent.

In a specific embodiment shown in FIG. 4, a carboxylic-acid-substituted maleimide compound 412, specifically, 4-maleimidobutyric acid (CAS #57078-98-5), is reacted with a multi-arm polymer 410, which comprises a core region that comprises a polyol residue R, for example, a tripentaerythritol polyol residue, and 8 hydroxyl-terminated polyethylene oxide arms where n ranges from 30 to 140, in an ester coupling reaction, for example, utilizing dicyclohexylcarbodiimide (DCC) or diisopropylcarbodiimide (DIC) as a carbodiimide coupling reagent, to form a multi-arm polymer 415 having a core region and 8 polyethylene oxide arms, each terminating in a maleimide containing moiety that is linked to the polyethylene oxide arms by an ester group, which can be hydrolyzed in vivo.

As previously noted, in various aspects, the present disclosure pertains to radiopaque products that comprise a reaction product of (a) an iodinated compound comprising one or more diene containing moieties, for example, one or more furan containing moieties, various examples of which are described above, and (b) a multi-arm polymer that comprises a plurality of dienophile containing moieties, for example, a plurality of maleimide containing moieties, various examples of which are also described above. Various examples of iodinated compounds comprising diene containing moieties and various examples of multi-arm polymers comprising dienophile containing moieties are described above.

In various embodiments, the radiopaque products the present disclosure are visible under fluoroscopy. In various embodiments, such radiopaque products have a radiopacity that is greater than 250 Hounsfield units (HU), beneficially anywhere ranging from 250 HU to 500 HU to 750 HU to 1000 HU or more (in other words, ranging between any two of the preceding numerical values). Such radiopaque products may be formed in vivo (e.g., using a delivery device like that described below), or such radiopaque products may be formed ex vivo and subsequently administered to a subject. Such radiopaque products can be used in a wide variety of biomedical applications, including medical devices, implants, and pharmaceutical compositions.

In addition to residues of an iodinated compound comprising one or more diene containing moieties and a multi-arm polymer comprising a plurality of dienophile containing moieties, the radiopaque products of the present disclosure can further comprise one or more additional agents.

Examples of such additional agents include therapeutic agents such anti-angiogenic agents, cytotoxic agents, chemotherapeutic agents, checkpoint inhibitors, immune modulatory cytokines, T-cell agonists, and STING (stimulator of interferon genes) agonists. Examples of such additional agents include imaging agents in addition to the iodine present in the radiopaque products.

Examples of such additional agents further include imaging agents, for example, (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) radiocontrast agents, such as those based on the clinically important isotope ^{99m}Tc, as well as other gamma emitters such as ¹²³I, ¹²⁵I, ¹³¹I, ¹¹¹In, ⁵⁷Co, ¹⁸³Sm, ¹³³Xe, ⁵¹Cr, ^{81m}Kr, ²⁰¹Tl, ⁶⁷Ga, and ⁷⁵Se, among others, (e) positron emitters, such as ¹⁸F, ¹¹C, ¹³N, ¹⁵O, and ⁶⁸Ga, among others, may be employed to yield functionalized radiotracer coatings, and (f) contrast agents for use in connection with near-infrared (NIR) imaging, which can be selected to impart near-infrared fluorescence to the coatings of the present disclosure, allowing for deep tissue imaging and device marking, for instance, NIR-sensitive nanoparticles such as gold nanoshells, carbon nanotubes (e.g., nanotubes derivatized with hydroxyl or carboxylic acid groups, for instance, partially oxidized carbon nanotubes), dye-containing nanoparticles, such as dye-doped nanofibers and dye-encapsulating nanoparticles, and semiconductor quantum dots, among others. NIR-sensitive dyes include cyanine dyes, squaraines, phthalocyanines, porphyrin derivatives and borondipyrromethane (BODIPY) analogs, among others.

When combined, a diene containing compound, such as a furan containing compound, and a dienophile containing compound, such as maleimide containing compound, will spontaneously and rapidly undergo a [4+2] Diels-Alder reaction thereby linking the diene containing compound to the dienophile containing compound. This reaction will proceed at room temp and increases with increasing temperature above room temperature (e.g., at 37° C.). In embodiments of the present disclosure, a diene containing iodinated compound, such as a furan containing iodinated compound, and a dienophile containing multi-arm polymer, such as a maleimide containing multi-arm polymer are combined such that they undergo a [4+2] Diels-Alder reaction, thereby linking the iodinated compound to the multi-arm polymer. Such reactions can be conducted in vivo or ex vivo. The highly reactive selectivity of the Diels-Alder reaction will only take place between the furan and maleimide groups, which avoids off-target or unintentional crosslinking in vivo.

As previously indicated, when the iodinated compound contains two or more furan containing moieties, [4+2] Diels-Alder reaction of the iodinated compound with the multi-arm polymer containing maleimide containing moieties results in furan-maleimide Diels-Alder adduct in the form of a crosslinked network wherein the iodinated compound is crosslinked with arms of the multi-arm polymer via

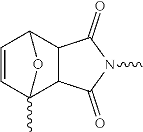

linkages.

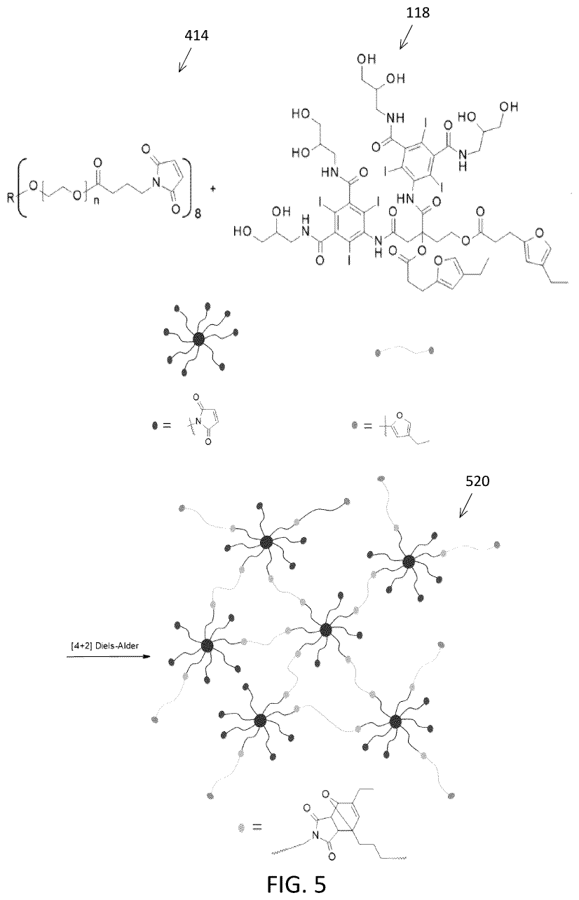

This can be seen, for example, in the schematic representation of FIG. 5, where the maleimide terminated multi-arm polymer 414 of FIG. 4 is coupled with the iodinated furan containing product 118 of FIG. 1B via a Diels-Alder reaction to form the crosslinked radiopaque product 520 shown.

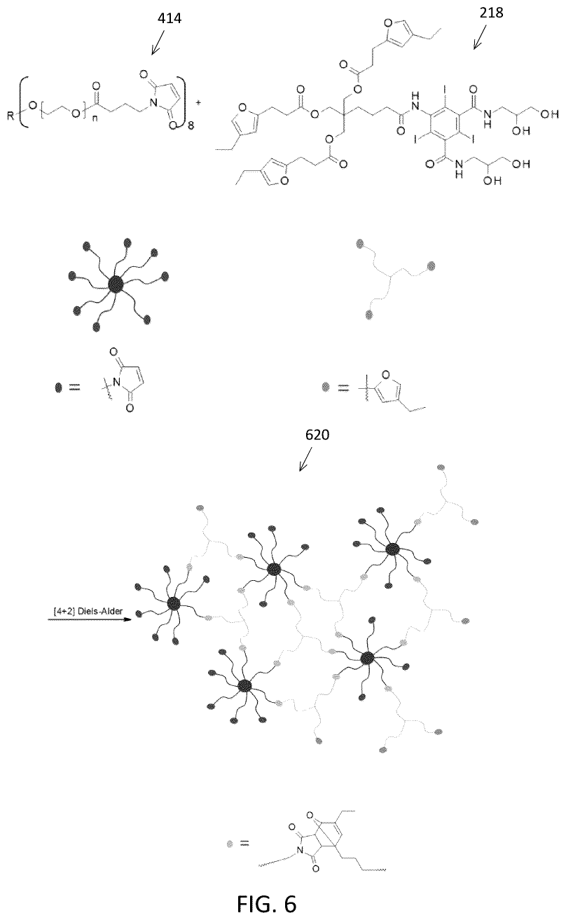

Similarly, in the schematic representation of FIG. 6, the maleimide terminated multi-arm polymer 414 of FIG. 4 is coupled with the iodinated furan containing product 218 of FIG. 2B via a Diels-Alder reaction to form the crosslinked radiopaque product 620 shown in FIG. 6.

On the other hand, wherein the iodinated compound contains only a single furan containing moiety, [4+2] Diels-Alder reaction of the iodinated compound with the multi-arm polymer containing maleimide containing moieties results in the coupling of the iodinated compound to the multi-arm polymer with no accompanying crosslinking.

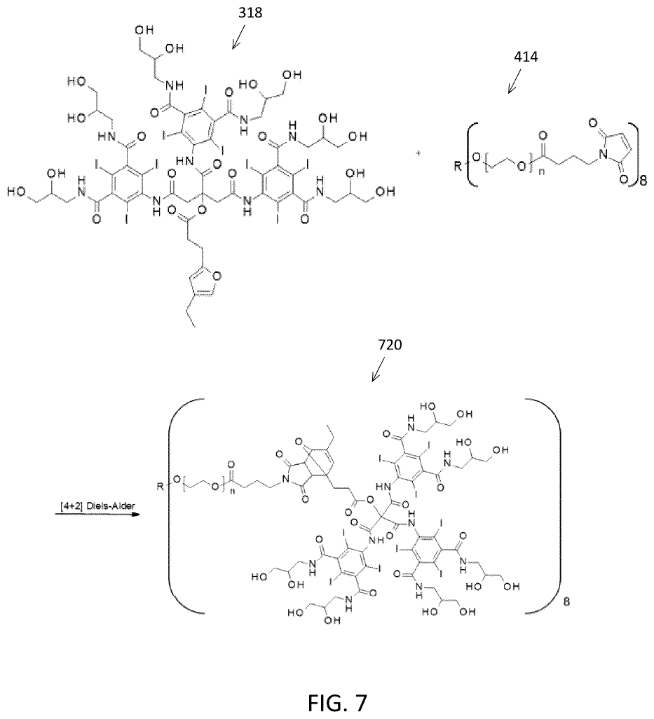

This is shown, for example, in FIG. 7, where the iodinated furan containing product 318 of FIG. 3B is coupled to the maleimide terminated multi-arm polymer 414 of FIG. 4 via a Diels-Alder reaction. Because the iodinated furan containing product 318 of FIG. 3B contains only a single furan group, the reaction is not a crosslinking reaction. However, the multi-arm polymer 414 is converted into a radiopaque product 720 by coupling it to the iodinated furan containing product 318.

In further aspects, the present disclosure pertains to systems that can be used to form crosslinked radiopaque products. The systems may comprise (a) a first composition comprising an iodinated multifunctional compound comprising a plurality of diene containing moieties and (b) and a second composition comprising a multifunctional multi-arm polymer that comprises a plurality of dienophile containing moieties. Various examples of iodinated compounds comprising diene containing moieties and various examples of multi-arm polymers comprising dienophile containing moieties are described above. The first and second compositions may be provided in first and second containers, respectively. For example, the first and second containers may be independently selected from vials and syringe barrels, among other formats.

In some aspects of the present disclosure, the systems are configured to dispense and combine the first and second compositions such that the iodinated multifunctional compound and the multifunctional multi-arm polymer crosslink with one another via a Diels-Alder reaction.

Such systems are advantageous, for example, in that the Diels-Alder reaction is highly selective, thereby minimizing off-target crosslinking. Such systems are also advantageous, for example, in that a buffer solution is not needed to maintain the pH at a particular value. Such systems are further advantageous, for example, in that iodine functionality, and thus radiopacity, is provided by the iodinated multifunctional compound that acts as a crosslinker for the multi-arm polymer. This allows reactive end groups to be provided on each of the polymer arms, thereby maximizing the crosslinking capacity of the multi-arm polymer, without sacrificing radiopacity.

The first composition may be a first fluid composition comprising the iodinated multifunctional compound or a first dry composition that comprises the iodinated multifunctional compound, to which a suitable fluid such as water for injection, saline, etc. can be added to form a first fluid composition. In addition to the iodinated multifunctional compound, the first composition may further comprise additional agents, including those described above.

The second composition may be a second fluid composition comprising the multi-arm polymer or a second dry composition that comprises the multi-arm polymer, to which a suitable fluid such as water for injection, saline, etc. can be added to form a second fluid composition. In addition to the multi-arm polymer, the second composition may further comprise additional agents including as those described above.

In various embodiments, a system is provided that include one or more delivery devices for delivering first and second compositions to a subject.

In some embodiments, the system may include a delivery device that comprises a first reservoir that contains a first composition that comprises an iodinated multifunctional compound as described above and a second reservoir that contains a second composition that comprises a multi-arm polymer as described above. During operation, the first composition and second composition are dispensed from the first and second reservoirs and combined, whereupon the iodinated multifunctional compound and the multi-arm polymer and crosslink with one another to form a hydrogel.

In particular embodiments, the system may include a delivery device that comprises a double-barrel syringe, which includes first barrel having a first barrel outlet, which first barrel contains the first composition, a first plunger that is movable in the first barrel, a second barrel having a second barrel outlet, which second barrel contains the second composition, and a second plunger that is movable in the second barrel.

In some embodiments, the device may further comprise a mixing section having a first mixing section inlet in fluid communication with the first barrel outlet, a second mixing section inlet in fluid communication with the second barrel outlet, and a mixing section outlet. In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique. Typically, the imaging techniques is an x-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A system for forming a radiopaque product that comprises (a) an iodinated compound comprising one or more diene containing moieties and (b) a multi-arm polymer comprising a plurality of dienophile containing moieties, wherein the diene containing moieties of the iodinated compound couple with the dienophile containing moieties of the multi-arm polymer by undergoing a Diels-Alder reaction.

2. The system of claim 1, wherein the iodinated compound comprises two or more diene moieties.

3. The system of claim 1, wherein the diene containing moieties are furan containing moieties and the dienophile containing moieties are maleimide containing moieties.

4. The system of claim 1, wherein the iodinated compound comprises a core, one or more furan containing moieties attached to the core, and one or more iodine containing moieties attached to the core.

5. The system of claim 4, wherein the one or more furan containing moieties are attached to the core though an ester group and the one or more iodine containing moieties are attached to the core through an amide group.

6. The system of claim 4, wherein the core is a residue of a hydroxy acid compound, wherein the one or more furan containing moieties are residues of a carboxylic-acid-substituted furan containing compound, and wherein the one or more iodine containing moieties are residues of an amino-substituted iodinated compound.

7. The system of claim 4, wherein the one or more iodine containing moieties comprise an aromatic structure that is substituted with one or more iodine groups.

8. The system of claim 7, wherein the aromatic structure is further substituted with one or more hydrophilic functional groups.

9. The system of claim 8, wherein the hydrophilic functional groups are selected from hydroxyl groups and C₁-C₄-hydroxyalkyl groups.

10. The system of claim 1, wherein the multi-arm polymer comprises a plurality of hydrophilic polymer arms.

11. The system of claim 10, wherein the hydrophilic polymer arms comprise one or more hydrophilic monomers selected from ethylene oxide, N-vinyl pyrrolidone, oxazolines, hydroxyethyl acrylate, hydroxyethyl methacrylate, PEG methyl ether acrylate or PEG methyl ether methacrylate, or N-isopropylacrylamide.

12. The system of claim 10, wherein two or more hydrophilic polymer arms of the plurality of hydrophilic polymer arms each comprise one or more dienophile end groups.

13. The system of claim 12, wherein the dienophile end groups are linked to the two or more hydrophilic polymer arms by a hydrolysable ester group.

14. The system of claim 1, comprising a first composition that comprises the iodinated compound comprising the one or more diene containing moieties in a first container and a second composition that comprises the multi-arm polymer comprising the plurality of dienophile containing moieties in a second container.

15. The system of claim 14, wherein the first and second containers are independently selected from vials and syringe barrels.

16. The system of claim 14, further comprising a delivery device.

17. A crosslinked network formed by combining an iodinated compound comprising one or more diene containing moieties with a multi-arm polymer comprising a plurality of dienophile containing moieties, wherein the diene containing moieties of the iodinated compound couple with the dienophile containing moieties of the multi-arm polymer by undergoing a Diels-Alder reaction.

18. The crosslinked network of claim 17, wherein the crosslinked network is a hydrogel.

19. The crosslinked network of claim 17, wherein the crosslinked network has a radiopacity that is greater than 250 Hounsfield units.

20. A method of treatment comprising administering to a subject a mixture that comprises an iodinated compound comprising the one or more diene containing moieties and a multi-arm polymer comprising the plurality of dienophile containing moieties, such that the diene containing moieties of the iodinated compound couple with the dienophile containing moieties of the multi-arm polymer by undergoing a Diels-Alder reaction after administration.
